(19) United States
(12) Patent Application Publication
(10) Pub. No.: US 2013/0224855 A1
(43) Pub. Date: Aug. 29, 2013

(54) CULTURE MEDIUM FOR EUKARYOTIC CELLS

(71) Applicants: Abhishek Gupta, Gujarat (IN); Mireille Maria Gadellaa, Bemmel (NL); Dominick Yves Willy Maes, Ede (NL)

(72) Inventors: Abhishek Gupta, Gujarat (IN); Mireille Maria Gadellaa, Bemmel (NL); Dominick Yves Willy Maes, Ede (NL)

(21) Appl. No.: 13/819,592

(22) PCT Filed: Aug. 31, 2011
(86) PCT No.: PCT/NL2011/050592

§ 371 Date: May 14, 2013

(30) Foreign Application Priority Data
Aug. 31, 2010 (EP) 10174715.2

(51) Int. Cl. C12N 5/00

(52) U.S. Cl. CPC C12N 5/0031
USPC 435/348; 435/404; 435/325

(57) ABSTRACT

The invention pertains to the use of amino acid derivatives selected from N-acetyl amino acids, γ-glutamyl amino acids, pyroglutamyl amino acids, glutamate-containing or proline-containing dipeptides, oxo-aminoacids, homo-aminoacids, and glycyl-glycine, as a growth- and production promoting ingredient, in culture media for culturing eukaryotic cells. The invention further pertains to culture media containing these amino acid derivatives at levels of at least 0.001 mg/l.

### FIELD OF THE INVENTION

The invention relates to the production of a medium for culturing eukaryotic, in particular animal cells, as well as to a cell culture medium thus produced and its use for in vitro cultivation of eukaryotic, in particular animals cells.

### BACKGROUND

The production of valuable biochemicals and biopharmaceuticals, for instance antibodies and antibiotics, by culturing mammalian, plant or insect cells requires proper culture media. Cell culture media formulations have been supplemented with a range of additives, including undefined components like fetal calf serum (FCS), several animal-derived proteins and/or protein hydrolysates of bovine origin.

Serum or serum-derived substances, such as albumin, transferrin or insulin, which are used in animal cell culture, may contain unwanted agents that can contaminate the cultures and the biopharmaceutical products obtained from these. Moreover, bovine derived protein products like bovine meat or collagen hydrolysates bear the risk of BSE contamination. Furthermore, additives derived from human serum have to be tested for all known viruses, including hepatitis and HIV that can be transmitted by serum.

In conclusion, all serum-derived products can be contaminated by unknown agents. In the case of serum or protein additives that are derived from human or other animal sources in cell culture, numerous problems (e.g. the varying quality and composition of different batches and the risk of contamination with viruses, mycoplasma or BSE) can occur. Therefore, plant protein hydrolysates or plant peptones are commonly used in culture media that should be free of animal components.

However, growth of animal cells in media without animal-derived cell culture additives is not always satisfactory. It is frequently observed that animal cells which are cultivated in vitro grow in lumps. This is considered to be a suboptimal condition as the cells in the core of the lump are deprived of nutrients and will die. There is also a risk of clogging the tubing or the Alters during downstream processing. The reduced viability of the cells can also be assessed by their appearance. Cells having a reduced viability show an irregular shape, i.e. a not-round shape, and in addition have a “granulated” cell content which is in contrast to healthy cells that have perfectly bright and transparent cell content.

WO 2006/123926 relates to a peptide composition for growing and/or culturing micro-organisms and/or cells on the basis of at least one vegetable protein source, preferably from rapeseed, wheat or caraway. The effect of wheat hydrolysate is addressed in the examples.

WO 2006/128764 discloses a process for cultivating mammalian cells producing complex proteins, wherein one or more plant-derived peptones are fed to the cell culture. Plant sources soy, cotton seed and pea are exemplified. The effect of soybean hydrolysate on cultivation of CHO cells is shown in the accompanying examples.

WO 2009/020389 discloses the use of a protein hydrolysate of Helianthus (sunflower) species as a constituent of a culture medium for culturing eukaryotic, in particular animal cells.

U.S. Pat. No. 5,534,538 relates to the use of N-acylated dipeptides, such as N-acetyl-alanyl-glutamine, in a cell culture medium containing fetal calf serum (FCS), that is more stable towards heat sterilization than non-acylated dipeptides. No effect on cell growth as compared to the non-acylated dipeptide and free amino acid equivalents was observed.

WO2009/033024A1 discloses the use in a cell culture medium of arginine-containing dipeptides and tripeptides obtained by fractionation of an animal-derived peptone.

EP2154244A1 relates to cell culture medium wherein the concentration of the amino acids serine as well as cysteine and/or tyrosine is maintained at a concentration of at least 1 mM.

US2003/0203448A1 describes a protein-free and serum-free medium for the cultivation of cells, comprising soy hydrolysate and optionally added free amino acids.

US2002/0039787 discloses a method for the in vitro culturing of microvascular endothelial cells, said method comprising culturing an enriched population of microvascular endothelial cells in the presence of an effective amount of human serum.

The functionality of the plant protein hydrolysates is a direct result of its chemical composition. It is affected by several factors like raw material, processing factors, process control and storage conditions. Therefore, it results in a persistent yet poorly studied phenomenon defined as “lot-to-lot variation”.

It is a major concern expressed by the biopharma industries, which is the customer of these hydrolysates, as it can mean variations in the product yields from 10 to 25% and it has direct financial consequences. The invention aims at relieving these concerns.

### SUMMARY OF THE INVENTION

It was found that certain low-molecular amino acid derivatives have a strong growth and production promoting effect on cell cultures of eukaryotic cells, especially animal cells in vitro. The presence of a minimum level of selected derivatives results in consistent and therefore commercially attractive production performance. Media containing these derivatives are excellently suitable for culturing eukaryotic, in particular animal cells. Thus the invention provides a cell culture medium containing such specific amino acid derivatives, as well as a process of producing these media and a method for cultivation of animal cells in vitro using compositions containing these amino acid derivatives as a medium constituent.

### DETAILED DESCRIPTION OF THE INVENTION

The invention pertains to a process of producing a culture medium for culturing eukaryotic cells, in particular animal cells, involving the use of one or more amino acid derivatives selected from N-acetyl amino acids, γ-glutamyl amino acids, pyroglutamyl amino acids, and glutamate-containing or proline-containing dipeptides, oxo-amino-acids, homo-aminoacids, and glycyl-glycine, as a growth-promoting or production-improving ingredient. The present invention also pertains to a medium for culturing eukaryotic, in particular animal cells, containing at least at least 0.02 ppm (0.02 mg/kg), preferably at least 0.2 mg/kg, more preferably at least 2 mg/kg, even more preferably at least 20 mg/kg, most preferably at least 50 ppm (50 mg/kg), on a dry weight basis, of one or more of the above amino acid derivatives.

Wherever in the present description amounts of ingredients of the cell culture medium of the invention are given on a dry weight basis, the final concentrations in the liquid medium can be derived by arbitrarily taking a dry solids content of 5% (50 g/l) and vice versa. Thus, an amount of 100 mg per kg of dry matter, corresponds, for the sake of deriving preferred levels, to 5 mg per l of the final liquid medium. This by no means implies that the dry solids content of the liquid medium should be 5%. Depending on the specific cell culture concentrations, dry solid levels of e.g. between 0.5 and 30 wt. %, preferably between 0.5 and 15 wt. %, more preferably between 1 and 15 wt. %, most preferably between 1 and 5 wt % can be chosen. The protein content (including amino acids and amino acid derivatives) of the liquid medium will typically be between 0.05 and 20.0 wt. %, preferably between 0.1 and 10.0 wt. %, more preferably between 0.1 and 7.5 wt. %, even more preferably between 0.1 and 1.0 wt %, most preferably between 0.15 and 0.75 wt. %.

The amino acid derivatives to be used according to the invention contain at least one up to three amino acid residues. In addition to one or two amino acid residues, they may contain functional groups, in particular acetyl groups or methoxy groups. The amino acid derivatives are relatively small molecules preferably having molar weights between 100 and 500 Da, more preferably between 120 and 400 Da.

Preferred groups of amino acid derivatives include:

(a) N-acetyl amino acids, preferably of single amino acids, particularly of the larger amino acids such as leucine, isoleucine, methionine, phenylalanine, tyrosine, tryptophan, ornithine, lysine, citrulline, arginine. Preferred N-acetyl amino acids are N-acetyl-methionine, N-acetyl-phenylalanine and N-acetyl-ornithine;
(b) Gamma-glutamyl amino acids, particularly of the larger aromatic amino acids phenylalanine, tyrosine, and tryptophan. Gamma-glutamyl derivatives are bound to the other amino acids by the γ-carboxyl group. Preferred γ-glutamyl amino acids are γ-glutamyl-tyrosine and γ-glutamyl-phenylalanine;
(c) Pyroglutamyl amino acids such as pyroglutamyl-glutamine and pyroglutamyl-glycine. Pyroglutamyl groups are glutamyl groups wherein the α-amino group is condensed with the γ-carboxyl group to form a cyclic group, and hence the pyroglutamyl group is a 5-oxopyrrolidin-2-ylcarbonylamino group;
(d) Glutamate-containing or proline-containing dipeptides such as valinyl-glutamate and glycylproline; cyclic dipeptides, such as cyclo-(glycyl-glutamate) are also included;
(e) Oxo-aminoacids, such as 5-oxoproline and S-oxo-methionine (methionine sulfoxide);
(f) Homo-aminoacids, wherein ‘homo’ means an addition of one methylene group in the main chain of a regular amino acid (one of the 20 amino acids directly obtainable by translation of genetic codes), such as β-alanine, homoserine, and 2-amino-butyrate (‘homo-alanine’).

Most preferred amino acid derivatives are γ-glutamyl-tyrosine and γ-glutamyl-phenylalanine, cyclo-glycyl-glutamate, valinyl glutamate, 5-oxoproline and β-alanine.

The amino acid derivatives to be used according to the invention can be used as such. Most of the components are commercially available. Alternatively, they can be produced by commonly known synthetic or semi-synthetic procedures. Most of the derivatives can also be isolated from suitable protein fractions or hydrolysates, especially plant-derived proteins such as from soybeans, peas, lentils, wheat (gluten), cottonseed, rice, sunflower, safflower etc. They can be extracted or enriched from the protein fraction, or more conveniently from protein hydrolysates. Such methods are known in the art, for example by Sato, Nisimura et al., Journal of Agricultural and Food chemistry 46(9): 3403-3405 (1998), Higaki-Sato, Sato, et al. Journal of Agricultural and Food chemistry 51: 8-13, (2003), and Morris and Thompson Biochemistry 1(4): 706-709 (1962).

The invention thus concerns a process of producing a cell culture medium by adding to further constituents of the medium an amount of one or more amino acid derivatives selected from N-acetyl amino acids, γ-glutamyl amino acids, pyroglutamyl amino acids, and glutamate-containing or proline-containing dipeptides, oxo-amino-acids, homo-aminoacids, and glycyl-glycine, such that the final concentration in the medium is at least 0.001 mg/l, preferably at least 0.01 mg/l, more preferably at least 0.1 mg/l, most preferably at least 1 mg/l per individual amino acid derivative, and as further elaborated below. It is preferred that the final concentration in the medium is at most 50 g/l, preferably at most 1 μl, more preferably at most 100 mg/l per individual amino acid derivative. The derivatives can be added as such, e.g. as purified and/or synthetic products, or as a concentrate, i.e. a product obtained by concentrating or enriching proteinaceous matter to a level of at least 1% by weight, preferably at least 2%, more preferably at least 5%, most preferably at least 10%, or even at least 25% by weight.

The invention further pertains to a cell culture medium obtainable by this process. More specifically, the invention relates to a culture medium for culturing eukaryotic cells containing at least 0.001 mg per 1, preferably at least 0.01 mg per 1, more preferably at least 0.1 mg per 1, even more preferably at least 1 mg per 1, most preferably at least 5 mg per 1 of final liquid medium of one or more amino acid derivatives selected from N-acetyl amino acids, γ-glutamyl amino acids, pyroglutamyl amino acids, glutamate-containing or proline-containing dipeptides, oxo-aminoacids, homo-amino acids, and glycyl-glycine, wherein the concentrations are per individual amino acid derivative. It is preferred that the final concentration in the medium is at most 50 preferably at most 1 g/l, more preferably at most 100 mg/l per said individual amino acid derivative. In terms of dry weight of the cell culture medium of the invention, it contains at least 0.02 mg per kg, preferably at least 0.2 mg per kg, more preferably at least 2 mg per kg, even more preferably at least 20 mg per kg, most preferably at least 250 mg per kg of dry matter, and at most 1000 g, preferably at most 20 g, more preferably at most 2 g per kg of dry matter of one or more amino acid derivatives selected from N-acetyl amino acids, γ-glutamyl amino acids, pyroglutamyl amino acids, glutamate-containing or proline-containing dipeptides, oxo-aminoacids, homo-amino acids, and glycyl-glycine.

In a preferred embodiment of the invention, a cell culture medium contains one or more of the above amino acid derivatives in a concentration of between 5 mg/l and 30 g/l, or between 100 mg and 600 g, preferably between 250 mg and 150 g per kg dry matter. More preferred levels are between 10 mg/l and 1 g/l or between 200 mg and 100 g, preferably between 500 mg and 50 g per kg dry matter, even more preferred between 20 mg/l and 500 mg/l or between 1 and 25 g per kg dry matter.

For N-acetyl amino acids, γ-glutamyl amino acids, and cyclo-glycyl-glutamine, the preferred level in a culture medium for culturing eukaryotic cells is at least 0.01 mg/l, preferably at least 5 mg per l, or at least 0.2 mg, preferably at least 100 mg, preferably 250 mg per kg of dry matter, more preferred 10 mg/l-10 g/l, even more preferred 10 mg/l-1 g/l, most preferred 20-400 mg/l(0.2-50, and 1-2 g/kg dry matter). For pyroglutamyl amino acids, glycyl-proline and glycyl-glycine, the preferred level is between 0.03 mg/l and 30 g/l (0.6 mg/kg-600 g/kg dry matter), preferably between 30 mg/l and 30 g/l, more preferably 30 mg/l and 3 g/l(0.6-600, preferably 1.5-150 g/kg dry matter), more preferred 50 mg/1-1 g/l (2.5-50 g/kg). For valinyl-glutamate, β-alanine, 2-aminobutyrate, oxo-amino acids and homo-amino acids, the preferred level is between 0.02 mg/l and 20 g/l (0.4 mg/kg-400 g/kg dry matter), preferably between 20 mg/l and 20 g/l, more preferably 20 mg/l and 2 g/l, most preferred 50-500 mg/l (400 mg-400 g/kg, preferably 1-100 g/kg and 2.5-25 g/kg dry matter).

In a particularly preferred embodiment of the invention, the amino acid derivatives are used as part of one or more vegetable protein hydrolysates. The protein hydrolysates can be produced by methods known in the art, e.g. by processing the beans, legumes, seeds etc. by pressing, grinding, dehulling and/or crushing, if desired followed by defatting, e.g. using organic solvents such as hexane. Preferably the defatted seed material contains at least 20 wt % protein. The defatted seed material preferably has a fat content of less than 10 wt. %.

A protein hydrolysate is usually obtained by enzymatic proteolysis and can also be referred to as proteolysate. The (defatted) plant seed material, optionally comminuted, is subjected to hydrolysis using endo and/or exo proteases from bacterial, fungal, vegetable or animal origin or mixtures thereof; however preferably the enzyme is not from an animal source. The enzyme may be produced using recombinant DNA techniques. The preferred enzymes are endo-proteases. More preferably the enzyme comprises alkaline proteases. Suitable proteases include a subtilisin (Alcalase), a serine endoprotease. Particularly suitable enzymes comprise Alcalase from Novozymes, and/or papain from Merck. Other suitable enzymes comprise e.g. Neutrase.

Hydrolysis conditions comprise a reaction time of between 30 minutes and 30 hours; preferably 1-6 hours, most preferably 2-4 hours; temperatures are between 20 and 65° C., preferably between 40° C. and 60° C., all depending on the particular protein source and the desired degree of hydrolysis. The pH may be adjusted between 6.0 and 8.5, preferably 6.6 and 8.0, most preferred is 7.0-8.0. The concentration of the protein to be hydrolysed in solution is between 1 and 10% protein, preferably 2-8, most preferably 3-6 wt. %. The amount of enzyme used is, based on substrate, between 0.5-10 wt %, preferably 1-5 wt %, most preferably 1.5-3.5 wt %.

The hydrolysis is preferably performed until a degree of hydrolysis of between 5 and 50%, preferably between 10 and 40%, most preferably between 10 and 30%, is attained. The hydrolysis reaction is terminated using a heat treatment. Preferably, the heat treatment encompasses a heating time of between 15 and 90 minutes between 80 and 100° C. (batch heat treatment), or 1-5 minutes at 100-120° C. Degree of hydrolysis may be determined using conventional formal titration, as demonstrated in the examples. After termination of the hydrolysis reaction, the reaction mixture can optionally be polished to remove insoluble parts, for example using centrifugation or filtering aids know in the art like diatomaceous earth (e.g. Celite®, Dicalite®, Hyflo®). Preferably, the hydrolysate contains less than 10 wt. %, on dry matter basis, of water-insoluble material, more preferably less than 5 wt. %, most preferably less than 2 wt. %. The hydrolysate can be dried, for instance by spray drying or freeze drying. The hydrolysate may be used as such or may be further fractionated.

The hydrolysate preferably contains between 20 and 80 wt. %, especially between 20 and 60 wt. % of peptides having a molecular weight of 100-500 Da and/or between 10 and 30 wt. % of peptides of a molecular weight between 500 an 1000 Da on total protein basis. In terms of peptide length, the hydrolysate preferably contains at least 15 wt. %, more preferably at least 25 wt. %, most preferably at least 35 wt. %, up to e.g. 85 wt. %, more preferably up to 65 wt. %, most preferably up to 55 wt. % of di- to penta-peptides, between 8 and 30 wt. % of hexa- to nonapeptides, at least 8 wt. %, especially between 15 and 60 wt. % of higher peptides and between 0.1 and 30 wt. %, preferably between 0.5 and 10 wt. % of free amino acids, on total protein basis. In a preferred embodiment, the hydrolysate may be ultrafiltered, preferably using a 5 or 10 kDa molecular weight cut-off. The hydrolysate may contain further constituents such as carbohydrates, soluble fibres, multivalent metal salts, etc. Preferably the protein content (all proteinaceous material including free amino acids) is between 30 and 90 wt. %, more preferably between 45 and 85 wt. %. These amounts are on a dry weight basis.

The hydrolysate may be combined with other conventional constituents of culture media such as plant or animal cytokines and/or growth factors (provided that these are not of animal origin), vitamins, minerals, amino acids, buffering salts, trace elements, nucleosides, nucleotides, phytohormones, sugars including glucose, antibiotics and the like. Phytohormones comprise auxins, gibberellins, abscisic acid and combinations thereof.

Also commercially available basal media may be used in combination with the amino acid derivatives of the invention and the protein hydrolysates. For an animal cell line as CHO-1, Power CHO-1 CD from Lonza, IS CHO-CD from Irvine Scientific, or Excell 325 PF CHO from SAFC may be used. For plant cells, Murashige and Skoog basal medium obtainable from SAFC may be used. The hydrolysate may also be a hydrolysate from different protein sources, such as hydrolysates from wheat and soy, soy and pea, rice and cottonseed, in any ratio which allows the amino acid derivatives to be present in the amounts given above. The cell culture medium preferably does not contain serum such as fetal calf serum, or serum-derived components in order to be full reproducible and/or to avoid contamination. Preferably, the cell-culture medium is free of animal components, such as animal-derived proteins and/or protein hydrolysates of animal, e.g. bovine, origin. Accordingly, in a preferred embodiment the invention pertains to a serum-free culture medium for culturing eukaryotic cells as defined herein, and to a process of preparing such a serum-free culture medium.

A compound analysis directed to a selection of the claimed amino acid derivatives present in a chemically defined, commercially available medium supplemented with soy protein hydrolysate as commonly known in the art is provided in the Examples section. From this analysis it is clear that the concentrations of these particular amino acid derivatives in hydrolysate-based or hydrolysate-enriched media as generally applied in the art are at least two orders of magnitude lower than those of the cell culture medium according to the present invention.

The cell culture medium and the method of culturing both according to the invention are capable of supporting cultivation of eukaryotic, in particular animal cells, where capability means that it enables at least the survival, proliferation and/or differentiation of—and preferably also the expression of product by the cells in vitro. Cultivation in batch, fed batch, continuous or perfusion reactors are all envisaged.

Cell growth curves can be separated in a real growth phase in which the cells multiply and grow, and a production phase, in which the cells are more or less in a steady state, but start to produce the metabolites of interest, e.g. antibodies. The amino acids derivatives of the invention are capable of supporting both the growth phase and the production phase of animal or other eukaryotic cells.

The cell culture medium may be provided as a liquid or in a powdered, dried form. The amount of (essentially water-soluble) hydrolysate in the liquid medium can be determined by the skilled person, but comprises preferably 0.01-10.0 wt/vol %, more preferably 0.01-4.0 wt/vol %, even more preferably 0.05-2.0 wt/vol %, or 0.05-1.0 wt/vol %, even more preferably 0.1-1.0 wt/vol %, and most preferably 0.2-0.6 wt/vol %.

The amount of hydrolysate in a dry culture medium that can be reconstituted with water is depending on the medium components, but is typically in the range of 2-80% w/w, preferably 5-50% w/w. The cell culture medium also preferably contains sugars, in particular glucose, preferably in a dry weight ratio of glucose to hydrolysate between 10 and 0.1, more preferably between 2.5 and 0.4, and further constituents as described above.

Furthermore, the invention concerns the use of the cell medium for culturing eukaryotic cells. Eukaryotes comprise Fungi (including yeasts), Protista, Chromista, Plantae and Metazoa (animals). The invention especially concerns the use for culturing plant cells, for example rice, tobacco and maize, and in particular animal cells, preferably in vitro cultivation. The cells to be cultured may be from a natural source or may be genetically modified. Animal cells especially comprise vertebrate and invertebrate cells, including mammalian cells such as human cells e.g. PER C6 Cells®, rodent cells, in particular Chinese Hamster Ovary (CHO) cells, avian, fish, reptile, amphibian or insect cells.

The cells cultured by the method of the invention are in particular used for expression of protein products that may be further purified in biopharmaceutical industry. Non-limiting examples of protein products that can advantageously be produced in the culture medium of the invention include erythropoietin (for treating blood disorders), etanercept (TNF-α inhibitor for treating rheumatic diseases and gout), alpha dornase (deoxyribonuclease for the treatment of cystic fibrosis), beta-interferon (for treating multiple sclerosis) and a wide range of therapeutic monoclonal antibodies.

The desired protein products may be recovered by methods known in the art, such as separating the cells from the culture medium and isolating the protein products from the cell-free liquid (supernatant) e.g. by fractionation, affinity chromatography (adsorption—desorption) or the like, or combinations thereof.

Furthermore, the invention concerns a kit comprising a fraction containing the amino acid derivatives, and one or more constituents of culture media selected from plant or animal cytokines and/or growth factors, vitamins, minerals, amino acids, buffering salts, trace elements, nucleosides, phytohormones, nucleotides, sugars and antibiotics. The constituents may be present in the kit as one or more combinations. For example, the amino acid derivatives may be separately present in dry or dissolved form and part or all of the further constituents of culture media such as plant or animal cytokines and/or growth factors, vitamins, minerals, amino acids, buffering salts, trace elements, nucleosides, nucleotides, phytohormones, sugars and antibiotics, may be present as a separate combination. Alternatively, the amino acid derivatives may be premixed with e.g. further amino acids and/or peptides and/or sugars, and any remaining constituents may be present separately or in one or more combinations. It is preferred that at least one of the compositions is a liquid, which liquid may advantageously be sterilised. The compositions of the kit are mixed prior to use of the culture medium.

It has thus been found that the amino acid derivatives according to the invention and their use have several important advantages. They have a growth promoting effect which exceeds the growth provided by common protein constituents. They result in enhanced production, a lower variance of production and/or growth, and are cost-effective.

Animal cells that are cultured in vitro are not growing in lumps or clusters but are present as single cells. Secondly, the viability of the cells is excellent as judged by their perfect round shape and bright transparent cell content. Thirdly, much higher cell densities can be obtained compared to state of the art cell culture media such as those based on non-serum protein, in particular soy protein, without compromising the expression level of the desired cell products. Fourthly, the hydrolysate can be combined with any basal culture medium for in vitro cultivation of animal cells, enabling the manufacture of a wide variety of cell culture media with the advantages mentioned above. Also the cultivation can be extended over prolonged periods, resulting in higher product yields.

### EXAMPLES

### Example 1

### Isolation of Gamma-Glutamyl Peptides from Soybeans

The method of Morris and Thompson, (1962) Biochemistry 1(4): 706-709, was followed: Yellow soybeans (25 kg) were powdered and thoroughly extracted at room temperature with 70% ethanol. The extracts were cooled to 5° C. and, after remaining at this temperature for several days, the clear supernatants were put through a Dowex 50 column (hydrogen form, 5° C.). Because the beans were not defatted before extraction, there was considerable precipitation of material on the resin columns. This material was not removed by the alcohol or water wash but was redissolved during elution with 2N ammonia.

The eluate was evaporated at 40° C. in vacuo, the residue dissolved in water, and the contaminant removed by precipitation at pH 4.0. The partially purified amino acids were absorbed on a 5.8×127 cm column of Dowex 1 Ac (200-400 mesh) and washed thoroughly with deionized water to remove neutral and basic amino acids. The initial eluent was 0.1N acetic acid, and 21-ml fractions were collected at a flow rate of 3.5 ml per minute. The normality of the acetic acid was changed to 0.3 at fraction 900, and to 1.0 at fraction 1400, and 2.0N acetic acid was introduced to the column at fraction 2400. One drop of solution from alternate fractions was placed, in rows, on a large sheet of filter paper, dried, and sprayed with a 0.5% solution of ninhydrin in ethanol.

The density of the colour indicated the tubes containing the peak amino acid concentrations. The peaks were then investigated by using small (18×18 cm two-directional chromatograms, which indicated that fractions 2000-2300 contained the glutamic-phenylalanine peptide and fractions 2630-2870 contained the glutamic-tyrosine peptide. Fractions 2000-2300 were combined, as were 2630 2870, the solvent was removed in vacuo, and the compounds were crystallized from water as colorless solids. After several recrystallizations, several hundred milligrams of each peptide were obtained as colourless crystals.

### Example 2

### Isolation of Pyroglutamyl Peptides of Wheat Gluten

The method of Sato et al., Journal of Agricultural and Food chemistry 46(9): 3403-3405. (1998) and Higaki-Sato et al. Journal of Agricultural and Food chemistry 51: 8-13 (2003) was followed:

Isolation of N-Terminal-Blocked Peptides: An AG50WX8 strong cation exchanger (Bio-Rad, Hercules, Calif.) was packed in a minispin column (10*5 mm, i.d., AB-1150, Atto, Tokyo, Japan). The column, which was successively prewashed with 50% methanol and distilled water, was equilibrated with 10 mM formic acid. Peptide sample (50 μg/100 μL) was applied to the minispin column. N-Terminal blocked peptides were eluted with 10 mM formic acid (100 mL*3 times).

Pyroglutamate Aminopeptidase Digestion: The N-terminal-blocked peptide fraction was digested with 1 mU of porcine liver pyroglutamate aminopeptidase (Takara, Kyoto, Japan) in 100 μL of the attached reaction buffer at 37° C. for 3 h. The reaction was terminated by adding 10 μL of formic acid.

### Example 3

### Preparation of Derivatised Amino Acids (Soy Hydrolysates)

The procedure of Leone-Bay, Journal of Medicinal chemistry 38: 4263-4269 (1995) was followed to prepare the acylated amino acids described herein. The preparation of N-cyclohexanoylphenylglycine is given as a representative example. Phenylglycine (50.0 g, 331 mmol) was dissolved with stirring in aqueous sodium hydroxide (414 mL, 2N) in an open flask. The resulting solution was cooled to about 10-15° C. in an ice/water bath, and cyclohexanecarbonyl chloride (44.2 mL, 331 mmol) was added dropwise, maintaining the reaction temperature at about 10-15° C. After the addition was complete, the reaction solution was stirred for 2.5 h at room temperature. The pH of the reaction mixture was adjusted to 9.5 with aqueous hydrochloric acid (37%), and the unreacted phenylglycine was separated as a white solid and removed by filtration. The pH of the filtrate was then further lowered to 4.5 and crude N-cyclohexanoylphenylglycine precipitated from solution. This solid was removed by filtration and recrystallized from methanol to give N-cyclohexanoyl-phenylglycine.

### Example 4

### Analysis of Protein Hydrolysates Containing Claimed Amino Acid Derivatives and Evidence of Growth Stimulation

Commercial plant protein hydrolysates like SE50MAF-UF, WGE80M-UF, CNE80M-UF, PCE80B obtained from FrieslandCampina Domo, USA were analysed by Liquid chromatography/Mass Spectrometry (LC/MS, LC/MS2) using a Waters Acquity UPLC and a Thermo-Finnigan LTQ mass spectrometer, which consists of an electrospray ionization (ESI) source and linear ion-trap (LIT) mass analyzer. The sample extract was split into two aliquots, dried, then reconstituted in acidic or basic LC-compatible solvents. One aliquot was analyzed using acidic positive ion optimized conditions and the other using basic negative ion optimized conditions in two independent injections using separate dedicated columns. Extracts reconstituted in acidic conditions were gradient eluted using water and methanol both containing 0.1% formic acid, while the basic extracts, which also use water/methanol, contain 6.5 mM ammonium bicarbonate. The MS analysis alternated between MS and data-dependent MS2 scans using dynamic exclusion. Biochemicals were identified by comparison to metabolomic library entries of purified standards or recurrent unknown entities. The combination of chromato-graphic properties and mass spectra gives an indication of a match to the specific compound or an isobaric entity. Thus, an overview of the biochemical components and their relative concentration present in plant protein hydrolysates was generated.

Furthermore, all the hydrolysates were tested in the cell culture assays for cell growth and antibody production.

Linear regression analysis was performed on the cell growth and compound analysis data in order to identify the biochemical components that significantly affected the cell growth and antibody production. Using the SLOPE function of Microsoft Excel 2003, the correlation between antibody production and relative concentration of the components was calculated, the results of which are presented in Table 1 below. The higher the positive slope, the higher the importance of a biochemical component in the cell culture. p-values, also calculated using MS Excel's correlation regression function, represent the significance of the values, with the lower the p-value (all between 0 and 1), the more significant the measured value.

TABLE 1
Correlation of antibody production and relative concentration
of amino acid derivates

| Biochemical component | SLOPE | p-value |
|---|---|---|
| gamma-glutamyltyrosine | 2825.11 | 0.000 |
| valinylglutamate | 2064.16 | 0.000 |
| beta-alanine | 1884.70 | 0.000 |
| 5-oxoproline | 1667.69 | 0.017 |
| cyclo(Gly-Glu) | 1584.22 | 0.000 |
| methionine sulfoxide | 1309.84 | 0.000 |
| N-acetylphenylalanine | 848.92 | 0.003 |
| alanylalanine | 176.50 | 0.000 |
| allo-threonine | −2.12 | 0.019 |
| aspartylphenylalanine | −15.81 | 0.000 |

### Example 5

### Preparation of Cell Culture Medium

The cell culture assay was carried out in commercially available IS CHO-CD medium (Irvine Scientific, Cat. No. 91119). To this media, L-Glutamine (2 mM), pluronic acid, hypoxanthine (100 μM) and thymidine (15 μM) were added. Penicillin and streptomycin were added to prevent any bacterial growth during the growth assay. The media was supplemented with β-Alanine, γ-glutamyl cysteine, glycyl-glycine, L-homoserine or N-acetyl methionine, all purchased from Sigma Aldrich, Germany, in varying concentrations (1×10⁻⁵ to 1×10⁻¹% (w/v), see Table 2). The supplemented medium was mixed with a vortex mixture, filtered using a 0.22 μm filter and subsequently used in a growth assay.

### Example 6

### IgG Production and Cell Growth

### In Vitro Cultivation of CHO Cells

### Cell Lines

An IgG expressing CHO cell line was used (CHO-2: ATCC CRL 11397, producing IgG4). The cell lines were grown in the adherent conditions for a few passages and once confluent, they were transferred to animal-free conditions in the supplemented media described in Example 5.

### Growth and Production Curves

To measure growth and production curves, Chinese hamster ovary (CHO) cells were grown in suspension culture in baffled flasks. 20×10⁶ cells were transferred in 25 ml media to the baffled flasks. Chemically defined media with and without added amino acid derivatives were tested. No fresh media was added during the growth assay. Cells were counted using the CEDEX HiRes cell counter (Innovatis, Germany). The cell counts were used to calculate the area under the growth curve and represented as dimensionless area under curve (AUC) values as described in detail in Ling, C. X, Huang, J. and Zhang, H. (2003), International joint conferences on artificial intelligence, pp. 329-341. The supernatant samples were taken every alternate days for the IgG production measurements. IgG production was measured using sandwich ELISA method. The specific IgG production was calculated by taking the ratio of cumulative IgG production (in mg/ml) and AUC measured at day 11 of the growth assay. The cells were visually inspected using a phase contrast microscope (Zeiss Axiovert 25, 400× magnification). The cell appearance was significantly improved when sufficient levels of the amino acid derivatives were present in the medium. Only single cells were observed and no aggregation of cells was seen. The cell shape was also positively affected. Cells had a much more round and bright appearance when cultured in medium containing sufficient levels of the amino acid derivatives. This was in contrast with the observation that a lot of cell aggregates were present in CHO cell cultures grown in chemically defined medium without amino acid derivatives.

TABLE 2
Specific IgG production and cell growth of CHO cells in chemically
defined cell culture medium (see Example 5 for details) with added
amino acid derivatives in varying concentrations. Production and
growth data in chemically defined cell culture medium without added
amino acid derivatives, as well as in chemically defined cell
culture medium supplemented with soy protein hydrolysate (0.4% w/v)
and with fetal calf serum (Gibco-Invitrogen; 5% v/v) are provided for
comparison.

| | | Specific IgG | Cell growth |
|---|---|---|---|
| | | production | (area under |
| | Concentration | after 11 | curve) after |
| Components added | (% w/v) | days | 14 days |
| β-alanine | 1 × 10⁻¹ | 92.8 | 62.5 |
| | 1 × 10⁻² | 108.6 | 51.3 |
| | 1 × 10⁻³ | 97.1 | 50.6 |
| γ-glutamyl cysteine | 1 × 10⁻¹ | 78.2 | 46.1 |
| | 1 × 10⁻² | 105.9 | 42.8 |
| | 1 × 10⁻³ | 68.33 | 58.0 |
| | 1 × 10⁻⁴ | 77.2 | 47.6 |
| | 1 × 10⁻⁵ | 72.5 | 58.9 |
| Glycyl-glycine | 1 × 10⁻¹ | 86.6 | 50.6 |
| | 1 × 10⁻² | 95.8 | 48.2 |
| | 1 × 10⁻³ | 104.2 | 49.6 |
| | 1 × 10⁻⁴ | 113.5 | 47.7 |
| | 1 × 10⁻⁵ | 102.0 | 51.0 |
| L-homoserine | 1 × 10⁻¹ | 122.0 | 45.7 |
| | 1 × 10⁻² | 72.0 | 59.1 |
| | 1 × 10⁻³ | 96.8 | 39.4 |
| N-acetyl methionine | 1 × 10⁻¹ | 91.9 | 48.6 |
| | 1 × 10⁻² | 90.4 | 47.9 |
| | 1 × 10⁻³ | 94.4 | 46.0 |
| | 1 × 10⁻⁴ | 90.3 | 38.6 |
| | 1 × 10⁻⁵ | 81.3 | 41.2 |
| None | N/A | 68.2 | 55.5 |
| Soy protein | N/A | 70.9 | 69.1 |
| hydrolysate (0.4% | | | |
| w/v) | | | |
| Fetal Calf Serum (5% | N/A | 41.9 | 217.6 |
| v/v) | | | |

### Example 7

### Analysis of Soy Protein Hydrolysate

A liquid chromatography-mass spectrometry (LC-MS) based method was used to determine the absolute concentrations of the amino acid derivatives L-homoserine, β-alanine, N-acetyl methionine, and glycyl-glycine in the hydrolysates. Pure L-homoserine, β-alanine, N-acetyl methionine, and glycyl-glycine were obtained from Sigma-Aldrich, Germany. The derivatives were diluted individually in Millipore water to obtain a concentration range. The LC-MS retention areas obtained for the diluted derivatives were plotted against the concentration of the respective components to obtain calibration curves. Subsequently, the peaks identified in the LC-MS spectra of soy protein hydrolysate (SE50MAF-UF, FrieslandCampina Domo) samples were related to the LC-MS peaks specific to the individual amino acid derivative components. Using the calibration curves for the individual components, the absolute amounts of the amino acid derivatives L-homoserine, β-alanine, N-acetyl methionine, and glycyl-glycine in the soy hydrolysate were calculated.

TABLE 3
Concentration of amino acid derivatives in typical cell growth medium
comprising chemically defined medium (IS CHO-CD medium
(Irvine Scientific, Cat. No. 91119) supplemented with 0.4% (w/v)
soy protein hydrolysate.

| Amino acid derivative | Concentration (% w/v) | Concentration (mg/L) |
|---|---|---|
| β-Alanine | 3.6 × 10⁻¹⁰ | 3.6 × 10⁻⁶ |
| L-homoserine | 9.2 × 10⁻¹⁰ | 9.2 × 10⁻⁶ |
| Glycyl-glycine | 3.8 × 10⁻¹¹ | 3.8 × 10⁻⁷ |
| N-acetyl methionine | 3.6 × 10⁻¹⁰ | 3.6 × 10⁻⁶ |

1-18. (canceled)

19. A process of producing a serum-free culture medium for culturing eukaryotic cells comprising:
(a) adding to conventional culture medium one or more amino acid derivatives selected from the group consisting of N-acetyl amino acids, γ-glutamyl amino acids, pyroglutamyl amino acids, glutamate-containing or proline-containing dipeptides, oxo-aminoacids, homo-amino acids, and glycyl-glycine, at a concentration of at least 0.001 mg/l per individual amino acid derivative,
wherein the one or more amino acid derivatives are added as a pure substance or as a concentrate, the concentrate having a concentration of at least 1 g of a derivative per 100 g proteinaceous matter.

20. The process according to claim 19, wherein the concentrate has a concentration of at least 10 g or a derivative per 100 g proteinaceous matter.

21. The process according to claim 19, wherein the N-acetyl amino acids are selected from the group consisting of N-acetyl-methionine, N-acetyl-phenylalanine and N-acetyl-ornithine.

22. The process according to claim 19, wherein the γ-glutamyl amino acids are selected from the group consisting of γ-glutamyl-tyrosine and γ-glutamyl-phenylalanine.

23. The process according to claim 19, wherein the pyroglutamyl amino acids are selected from the group consisting of pyroglutamyl-glutamine and pyroglutamyl-glycine.

24. The process according to claim 19, where the glutamate-containing or proline-containing dipeptides are selected from the group consisting of valinyl-glutamate, glycyl-proline and cycloglycyl-glutamine.

25. The process according to claim 19, wherein the oxo-aminoacids are selected from the group consisting of 5-oxoproline and S-oxo-methionine.

26. The process according to claim 19, wherein the homo-amino acids are selected from the group consisting of (3-alanine, 2-aminobutyrate and homoserine.

27. A cell culture medium, obtained by the process according to claim 19.

28. The cell culture medium according to claim 27, comprising one or more of the amino acid derivatives in a concentration of at least 0.001 mg/l.

29. A culture medium for culturing eukaryotic cells comprising at least 0.001 mg per 1 of one or more amino acid derivatives selected from N-acetyl amino acids, γ-glutamyl amino acids, pyroglutamyl amino acids, glutamate-containing or proline-containing dipeptides, oxo-aminoacids, homo-amino acids, and glycyl-glycine.

30. The culture medium according to claim 29, comprising at least 5 mg per 1 of the one or more amino acid derivatives.

31. The culture medium according to claim 29, comprising at least 0.02 mg per kg of the one or more amino acid derivatives per kg of dry matter.

32. The culture medium according to claim 29, comprising at least 250 mg per kg of the one or more amino acid derivatives per kg of dry matter.

33. The culture medium according to claim 29, comprising between 0.01 mg/l and 10 g/l of one or more amino acid derivatives selected from N-acetyl amino acids, γ-glutamyl amino acids, and cyclo-glycyl-glutamine.

34. The culture medium according to claim 23, comprising between 10 mg/l and 1 g/l of one or more amino acid derivatives selected from N-acetyl amino acids, γ-glutamyl amino acids, and cyclo-glycyl-glutamine.

35. The culture medium according to claim 29, comprising between 0.03 mg/l and 30 g/l of one or more amino acid derivatives selected from pyroglutamyl-glycine, glycyl-proline and glycyl-glycine.

36. The culture medium according to claim 35, comprising between 30 mg/l and 3 g/l of one or more amino acid derivatives selected from pyroglutamyl-glycine, glycyl-proline and glycyl-glycine.

37. The culture medium according to claim 29, comprising between 0.02 mg/l and 20 g/l of one or more amino acid derivatives selected from valinyl glutamate, β-alanine, 2-aminobutyrate, oxo-amino acids and homo-amino acids.

38. The culture medium according to claim 37, comprising between 20 mg/l and 2 g/l of one or more amino acid derivatives selected from valinyl glutamate, β-alanine, 2-aminobutyrate, oxo-amino acids and homo-amino acids.

39. A method of culturing eukaryotic cells in vitro, comprising growing the cells in a culture medium according to claim 29.

40. The method according to claim 39, wherein the eukaryotic cells comprise mammalian and/or insect cells.
